(19) United States
(12) Patent Application Publication
(10) Pub. No.: US 2005/0260577 A1
(43) Pub. Date: Nov. 24, 2005

(54) Detection of neurodegenerative disorders

(76) Inventors: Kay Double, Parramatta (AU); Dominic B. Rowe, Hornsby (AU); Manfred Gerlach, Hammelburh (DE); Peter Riederer, Wurzberg (DE)

Correspondence Address: Randolph Ted Apple;Morris & Foerster, 755 Page Mill Road, Palo Alto, CA, 94304-1018, US

(21) Appl. No.: 10/398,789

(22) PCT Filed: Oct. 9, 2001
(86) PCT No.: PCT/AU01/01271

§ 371 Date: Dec. 19, 2003

(30) Foreign Application Priority Data
Oct. 9, 2000 (AU) PR 0632
Sep. 13, 2001 (AU) PR7666

(51) Int. Cl. C12Q 1/68; G01N 33/53; G01N 33/567

(52) U.S. Cl. 435/6; 435/7.2

(57) ABSTRACT

The present invention relates to methods of detecting neurodegenerative diseases or disorders, particularly to methods for early detection of neurodegenerative diseases, such as Parkinson's disease. The present invention provides a method for detecting a neurodegenerative disease in a subject, the method comprising testing the subject for an indicator of release of neuromelanin from cells in the brain, wherein a positive test is indicative of death of brain cells containing neuromelanin and is characterised by an elevated level of the indicator of release of neuromelanin compared to control values.

### TECHNICAL FIELD

The present invention is directed generally to methods of detecting neurodegenerative diseases or disorders, particularly to methods for early detection of Parkinson's disease.

### BACKGROUND ART

Parkinson's disease (PD) is a progressive neurodegenerative disease characterised by motor dysfunction. A clinical diagnosis is made on the basis of a triad of motor symptoms: tremor, rigidity and bradykinesia or slowness of movement. Pathologically, PD is characterised by the death of the neurons in an area of the brain called the substantia nigra (SN) which produce the neurotransmitter dopamine (DA).

A major focus of current research is to identify strategies by which the death of these neurons can be slowed or halted, thus curbing the progression of the disease. It is likely that such strategies, termed “neuroprotection”, will be maximally efficacious when the proportional loss of dopaminergic neurons is relatively low. Post-mortem (Fearnley and Lees, 1991) and imaging studies (Morrish et al. 1998) suggest, however, that the rate of loss of dopaminergic neurons throughout the disease course is not linear but decelerates exponentially. Dopaminergic neuron death occurs rapidly during the first 7 years of the disease resulting in the loss of at least 65% of total SN dopaminergic neuron number during this period, although average neuron loss is much greater (Halliday et al., 1996). As the brain has a substantial capacity for compensation no motor symptoms are evident at this time. Thus, this period of rapid neuron loss in the absence of clinical signs is termed “preclinical disease”. Neurological signs, and thus a clinical diagnosis, occurs only after at least 65% of the neurons are lost, thus the majority of neurodegeneration in this disease occurs before the clinical onset and diagnosis of the disease.

The rate of loss of the remaining 35% of the neurons proceeds at a significantly slower rate during the remaining 10 to 20 years of the disease, the so-called “clinical phase” (FIG. 1). The pattern of cell loss presents a challenge in that the time of the most rapid cell loss, which represents the optimal time-point for the initiation of neuroprotective strategies, occurs in the absence of an identifiable clinical syndrome.

Accordingly, there is a need to develop a means for detecting a neurodegenerative disease in a subject, and preferably a means for detecting a neurodegenerative disease during this “preclinical” phase.

The present invention involves the detection of an indicator of the release of a substance called neuromelanin (NM) from the cytoplasm of the dying cells. NM is a complex polymer pigment believed to be formed from oxidized dopamine products within the dopaminergic neurons of the SN (and the noradrenergic neurons of the locus ceruleus (LC)). NM usually occurs as granules which can be seen in the cell body but as a consequence of cell death NM is released into the extracellular space (FIG. 2).

Therefore, the present invention provides a method for detecting a neurodegenerative disease in a subject, the method comprising testing the subject for an indicator of release of neuromelanin from cells in the brain, wherein a positive test is indicative of death of brain cells containing neuromelanin and is characterised by an elevated level of the indicator of release of neuromelanin compared to control values. The identification of a specific marker for the death of these melanised cells provides a means for detecting the disorders characterised by the death of these cells, even prior to the onset of clinical symptoms.

### SUMMARY OF INVENTION

In a first aspect, the present invention provides a method for detecting a neurodegenerative disease in a subject, the method comprising testing the subject for an indicator of release of neuromelanin from cells in the brain, wherein a positive test is indicative of death of brain cells containing neuromelanin and is characterised by an elevated level of the indicator of release of neuromelanin compared to control values.

In a second aspect of the invention, there is provided a method to detect a neurodegenerative disease in a subject who is tested prior to having any clinical symptoms of said neurodegenerative disease, wherein said method comprises testing the subject for an indicator of release of neuromelanin from cells in the brain, wherein a positive test is indicative of death of brain cells containing neuromelanin and is characterised by an elevated level of the indicator of release of neuromelanin compared to control values.

Typically, cell death is associated with the neurodegenerative disease in the subject.

Typically, the clinical symptoms of the neurodegenerative disease are the classical symptoms of such a disease. More typically, these symptoms include tremor, rigidity and bradykinesia or slowness of movement.

Preferably, the indicator is an immune response in the form of circulating antibodies to neuromelanin (NM), or analogues thereof, in the subject.

In a preferred form, the method according to the first or second aspect of the present invention employs the detection of antibodies capable of reacting to NM, or an antigenic fragment or analogue thereof, present in a subject.

Preferably, the analogue of NM is selected from the group consisting of synthetic dopamine melanin and synthetic noradrenaline melanin.

In a preferred form, the method of the first or second aspect comprises:
(i) obtaining a blood sample from said subject;
(ii) isolating sera from said blood;
(iii) incubating said sera with an antigen selected from human NM or synthetic dopamine melanin under conditions suitable for antibody-antigen binding; and
(iv) detecting bound antibody.

Typically, detection of bound antibody utilises labelled anti-human IgG.

In a third aspect, the present invention provides a method of treatment of a neurodegenerative disease in a subject, the method comprising:
(a) screening subjects to detect a neurodegenerative disease using the method according to the first or second aspect of the present invention; and
(b) initiating treatment for subjects tested positive for the indicator of release of neuromelanin from cells in the brain.

Typically, the treatment includes, but is not limited to, administering a therapeutically effective amount of at least one of the following: antioxidants, iron chelators, nonamine oxidase inhibitors, apoptosis inhibitors, growth factors, dopamine receptor inhibitors, endogenous enzymes which protect against oxidative damage such as glutathione, superoxide dismutase and catalase, inhibitors of excitatory damage, zonisamide, benzamide compounds, or ethanesulfonyl-piperidine derivatives, or a combination thereof.

In a fourth aspect, the present invention provides a system for the detection of a neurodegenerative disease in a subject, the system comprising:
(a) means for capturing an indicator of release of neuromelanin from cells in the brain of a subject; and
(b) means for detecting the captured indicator of release of neuromelanin from cells in the brain.

When the indicator of release of neuromelanin is circulating antibodies in a subject, the means for capturing the indicator of release of NM is preferably neuromelanin or an antigenic fragment or analogue thereof immobilized to a solid surface. In this situation, a detectably labelled probe is applied which is specific for antibodies captured on the solid support via the neuromelanin, fragment or analogue. The means for detecting the captured indicator can be any suitable means for detecting the presence of the label on the probe.

In a preferred form, the system according to the fourth aspect of the present invention is a kit comprising neuromelanin or an antigenic fragment or an analogue thereof bound to a solid support and a source of a detectably labelled probe, wherein, in use, a subject's serum sample is applied to the solid support such that any antibodies to neuromelanin will bind to the support via the neuromelanin or an antigenic fragment or analogue thereof, a sample of the probe is added and allowed to bind to the bound antibody, and the label on the bound probe is detected.

Typically, the probe is a labelled antihuman IgG.

More typically, the probe is horseradish peroxidase-conjugated goat antihuman IgG.

Typically, the components of the kit are housed in separate containers.

Typically, the kit may further comprise one or more other containers, containing other components, such as wash reagents, and other reagents capable of detecting the presence of bound antibodies. More typically, the detection reagents may include reagents capable of reacting with the labelled probe.

An ELISA-based system is particularly suitable for this aspect of the invention.

In a fifth aspect, the present invention relates to the use of the system according to the fourth aspect of the present invention to detect a neurodegenerative disease in a subject.

Typically, a method for detecting a neurodegenerative disease in a subject using the system according to the fourth aspect of the present invention comprises testing the subject for an indicator of release of neuromelanin from cells in the brain, the method comprising
(a) contacting a serum sample of said subject to the solid support of the system,
(b) adding sample of the detectably labelled probe of the system, and
(c) detecting probe bound antibody within said serum sample, wherein a positive test is indicative of death of brain cells containing neuromelanin and is characterised by an elevated level of the indicator of release of neuromelanin compared to control values.

In a sixth aspect, the present invention relates to human NM or an antigenic fragment or analogue thereof when used to detect a neurodegenerative disease in a subject.

In a seventh aspect, the present invention relates to human NM or an antigenic fragment or analogue thereof when used to detect a neurodegenerative disease in a subject who is tested prior to having any clinical symptoms of said neurodegenerative disease.

Typically, for the purposes of the first through seventh aspects of the invention, the neurodegenerative disease is selected from the group consisting of idiopathic Parkinson's disease, and parkinsonism including Multisystem Atrophy, Progressive Supranuclear palsy, Pick's disease, Corticobasal Degeneration and Dementia with Lewy Bodies. More preferably, the disease is idiopathic Parkinson's disease.

### Definitions

As used herein the term Parkinson's disease (PD) is used to indicate both idiopathic PD and the associated parkinsonian syndromes characterised by dopaminergic degeneration within the SN. The associated syndromes known under the umbrella term of the parkinsonian syndromes include multisystem atrophy and progressive supranuclear palsy, and share both some pathological and clinical features with the idiopathic Parkinson's disease.

The term “analogue” as used herein with reference to neuromelanin means a synthetic neuromelanin, or fragment thereof, which has the characteristic features of neuromelanin.

As used herein the term “neurodegenerative disease” refers to a disease which is characterised by degeneration of neuromelanin-containing neuronal cells. Where the disease includes a preclinical phase the term neurodegenerative disease will be understood to include the preclinical phase as well as, in all cases, to include the clinical phase of the disease.

As used herein the term “treatment”, refers to any and all uses which remedy a disease state or symptoms, or otherwise prevent, hinder, retard, or reverse the progression of disease or other undesirable symptoms in any way whatsoever.

The term “therapeutically effective amount” as used herein, includes within its meaning a non-toxic but sufficient amount a compound or composition for use in the invention to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In the context of this specification, the term “comprising” means “including principally, but not necessarily solely”. Furthermore, variations of the word “comprising”, such as “comprise” and “comprises”, have correspondingly varied meanings.

Any description of prior art documents herein, or statements herein derived from or based on those documents, is not an admission that the documents or derived statements are part of the common general knowledge of the relevant art in Australia or elsewhere.

In order that the present invention may be more clearly understood preferred forms will be described with reference to the following examples and drawings.

### BRIEF DESCRIPTION OF THE DRAWINGS

FIG. 1 is a schematic representation of the progression of cellular pathology of Parkinson's disease.

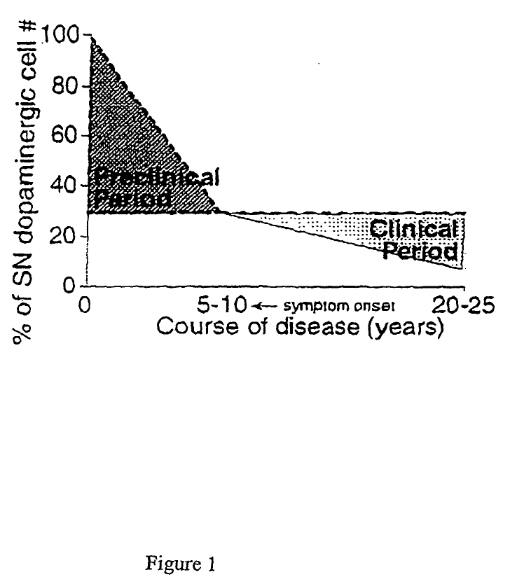

FIG. 2 is a photomicrograph of extracellular neuromelanin (arrow heads) and intracellular neuromelanin (arrow) in a brain of a Parkinson's disease sufferer.

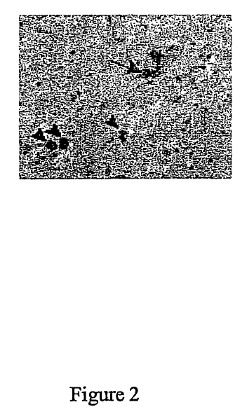

FIG. 3: Immune response in Parkinson's disease (PD), healthy age- and sex-matched control individuals without a family history of PD and individuals suffering depression. The response in the PD group is significantly higher than both the unrelated control group (p=0.005) and the depression group (p=0.046).

FIG. 4 shows the results of the cross-reactivity of IgG responses to a number of melanin analogues.

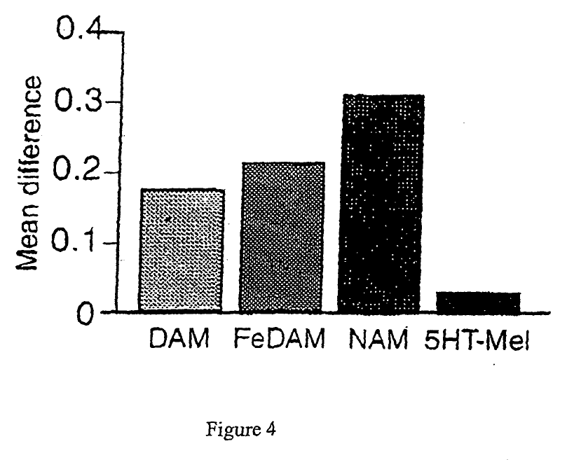

### DETAILED DESCRIPTION OF THE INVENTION

The present invention is concerned with the general class of neurodegenerative diseases, especially those generally classified under the umbrella term of “Parkinson's disease”, including idiopathic Parkinson's disease, and the parkinsonian diseases Multisystem Atrophy, Progressive Supranuclear palsy, Pick's disease, Corticobasal Degeneration and Dementia with Lewy Bodies.

Each of these diseases share the neuropathological characteristic that the NM-containing neurons of the substantia nigra degenerate and thus release NM. Other areas of the brain may also degenerate. However because degeneration of the cells of the substantia nigra is typical of each of these diseases, and hence the release of NM is typical, each of the aforementioned neurodegenerative diseases may be detected by detection of an indicator of release of NM.

Release of NM into the extracellular spaces, by degeneration of dopaminergic cells of the SN, or other areas of the brain such as the LC, exposes the NM to the immune system of the individual. An immune response is elicited and detection of that immune response provides an indication of release of NM through cell death.

In classical or idiopathic Parkinson's disease at least 65% of total SN dopaminergic neurons are lost prior to onset of the classical clinical symptoms of the disease. The triad of motor symptoms, tremor, rigidity and bradykinesia typify the onset of the clinical phase of the disease during which the rate of loss of the remaining 35% of dopaminergic cells is significantly slower than during the preclinical phase.

A humoral response to NM can be detected in a peripheral tissue, such as blood.

Accordingly, the present inventors describe an improved means for diagnosing a neurodegenerative disease which may be used, for example, independently in diagnosis of a disease or to confirm a diagnosis made on the basis of clinical symptoms. By providing a means which is independent of clinical symptoms the present invention also makes possible the diagnosis of neurodegenerative disease, in particular Parkinson's disease, before the onset of clinical symptoms. That is, by providing a means of diagnosis of a pathological occurrence in a neurodegenerative disease rather than diagnosis based on clinical symptoms, the method makes possible diagnosis in the pre-clinical phase of the neurodegenerative disease, such as Parkinson's Disease.

The method for detecting a neurodegenerative disease in a subject involves testing the subject for an indicator of release of neuromelanin from cells in the brain. The method involves detection of an indirect indicator of the release of neuromelanin (NM) from cells in the brain which can be detected in subjects with clinical or preclinical stages of neurodegenerative disease.

The indicator of release of NM may be circulating antibodies capable of binding to NM. The antibodies capable of binding to NM may also be capable of binding to an antigenic fragment of NM or an analogue of NM. Preferably, the analogue of NM is selected from the group consisting of synthetic dopamine melanin and synthetic noradrenaline melanin.

A positive test is indicative of death of brain cells containing neuromelanin and is characterised by an elevated level of the indicator of release of neuromelanin compared to control values. Control values are determined by testing healthy individuals having no indications of motor dysfunction. The individuals may be age- and sex-matched for the individuals being tested.

An elevated level of the indicator of release of NM reflects death of dopaminergic neurons, which is associated with a neurodegenerative disease in the subject.

In the process of detection a sample of a body fluid or tissue, containing an indicator of release of NM, is obtained from a test subject. Preferably the tissue is blood. The skilled person will recognise that the sample may be used in the performance of the method immediately or may be stored under suitable conditions until required.

Where the sample is a blood sample, serum may be isolated by standard methods. The sera may be used immediately or stored under suitable conditions, for example at −80° C., until required.

A method of detection involves an antibody capable of reacting with neuromelanin, or an antigenic fragment or analogue thereof, and includes an antibody which is capable of specifically binding with the neuromelanin, or antigenic fragment or analogue thereof. An antibody is capable of specifically binding with an antigen if it exhibits a threshold level of binding activity and/or it does not significantly cross-react with unrelated antigens. Antibodies herein specifically bind if they bind to said species with a binding affinity (Ka) of 10⁵ mol⁻¹ or greater, typically 10⁶ mol⁻¹ or greater, preferably 10⁷ mol⁻¹ or greater, more preferably 10⁸ mol⁻¹ or greater, and even more preferably 10⁹ mol⁻¹ or greater. The binding affinity of an antibody can be determined, for example, by Scatchard analysis (G. Scatchard, Ann. NY Acad. Sci. 51, 660-672, 1949).

Detection of the antibodies can utilise any known means of detecting antibodies including radioimmunoassays, enzyme-linked immunosorbant assays, solid phase assays.

In radioimmunoassay, C14-labelled synthetic dopamine melanin is synthesized and immunoprecipitation methods used to detect limiting dilution concentrations of antibody to the antigenic determinant.

Preferably, the method utilises the enzyme-linked immunosorbant assay (ELISA) using NM isolated from the human brain or synthetic dopamine melanin as the antigen. However, solid-phase immunoassay using synthetic neuromelanin immobilized on polystyrene and assayed by optical agglutination techniques to determine limiting dilution provides an alternate method of detection.

These methods are routinely employed in the detection of antigen-specific antibody responses, and are well described in general immunology text books such as Immunology by Ivan Roitt, Jonathan Brostoff and David Male (London: Mosby, c1998. 5th ed. and Immunobiology: Immune System in Health and Disease/Charles A. Janeway and Paul Travers. Oxford: Blackwell Sci. Pub., 1994), the contents of which are herein incorporated by reference.

Further, in terms of treatment of a neurodegenerative disorder, therapeutic agents are often present in the form of pharmaceutical and/or therapeutic formulations, that is, therapeutic agents present together with a pharmaceutically acceptable carrier, adjuvant and/or diluent.

Typically therapeutic agents include: antioxidants, iron chelators, nonamine oxidase inhibitors, apoptosis inhibitors, growth factors, dopamine receptor inhibitors, endogenous enzymes which protect against oxidative damage such as glutathione, superoxide dismutase and catalase, inhibitors of excitatory damage, zonisamide, benzamide compounds, or ethanesulfonyl-piperidine derivatives, or a combination thereof.

A preferred therapeutic agent is zonisamide, or an alkali metal salt thereof, and in relation to this, the disclosure of European Patent Application No. EP 1 040 830 is incorporated herein by reference.

Another preferred therapeutic agent is selected from the benzamide group of compounds, and in relation to this, the disclosure of U.S. Pat. No. 6,140,369 is incorporated herein by reference. Typical therapeutic agents from the benzamide group of compounds are selected from the group consisting of: N-tert-butyl-4-acetamidobenzamide, N-iso-propyl-4-acetamidobenzamide, N-tert-amyl-4-acetamidobenzamide, N-tert-butyl-3-acetamidobenzamide, N-methylcyclopropyl-4-acetamidobenzamide, N-n-butyl-4-acetamidobenzamide, N-n-pentyl-2-acetamidobenzamide, N-tert-butyl-2-acetamidobenzamide, N-iso-butyl-4-acetamidobenzamide, N-n-propyl-4-acetamidobenzamide, N-n-propyl-4-acetamidobenzamide, N-1,2-dimethylpropyl-4-acetamidobenzamide, N-n-pentyl-4-acetamidobenzamide, N-2-methylbutyl-4-acetamidobenzamide, N-tert-butyl-2,3-diacetamidobenzamide, N-tert-amyl-2,4-diacetamidobenzamide, N-tert-butyl-2,5-diacetamidobenzamide, N-tert-butyl-2,6-diacetamidobenzamide, N-tert-butyl-3,4 diacetamidobenzamide, N-tert-butyl-3,5-diacetamidobenzamide, N-iso-propyl-4-nitrobenzamide, N-tert-butyl-3-nitrobenzamide, N-tert-butyl-2-nitrobenzamide, N-n-butyl-4-nitrobenzamide, N-n-propyl-4-nitrobenzamide, N-tert-butyl-3,5-nitrobenzamide, N-tert-amyl-4-nitrobenzamide, N-1,2-dimethylpropyl-4-nitrobenzamide, N-n-butyl-3-nitrobenzamide, N-n-butyl-3,5-dinitrobenzamide, N-methylcylopropyl-4-nitrobenzamide, N-n-butyl-2-nitrobenzamide, N-n-pentyl-4-nitrobenzamide, N-2-methylbutyl-4-nitrobenzamide, N-n-pentyl-2-nitrobenzamide, N-1-methylpropyl-4-nitrobenzamide, N-tert-butyl-3-aminobenzamide, N-tert-butyl-4-aminobenzamide and N-methylcylopropyl-4-aminobenzamide, or a combination thereof.

A still further preferred therapeutic agent is selected from the ethanesulfonyl-piperidine derivative group of compounds, and in relation to this, the disclosure of international (PCT) Publication No. WO 00/75109 is incorporated herein by reference. Typical therapeutic agents from the ethanesulfonyl-piperidine derivative group of compounds are selected from the group consisting of: 4-[-2-(4-benzyl-piperidine-1-yl)-ethanesulfonyl]-phenol; 4-[2-(4-p-tolyloxy-piperidine-1-yl)-ethanesulfonyl]-phenol; (−)-(3R,4R)- or (3S,4S)-4-benzyl-1-[2-(4-hydroxy-benzenesulfonyl)-ethyl]-piperidin-3-ol; (+)-(3S,4S)- or (3R,4R)-4-benzyl-1-[2-(4-hydroxy-benzenesulfonyl)-ethyl]-piperidin-3-ol; (3RS,4RS)-4-benzyl-1-[2-(4-hydroxy-benzenesulfonyl)-ethyl]-piperidin-3-ol; (−)-(3R,4R)- or (3S,4S)-1-[2-(4-hydroxy-benzenesulfonyl)-ethyl]-4-(4-methyl-benzyl)-piperidin-3-ol; (+)-(3R,4R)- or (3S,4S)-1-[2-(4-hydroxy-benzenesulfonyl)-ethyl]-4-(4-methyl-benzyl)-piperidin-3-ol; and (3RS,4RS)-1-[2-(4-hydroxy-benzenesulfonyl)-ethyl]-4-(4-methyl-benzyl)-piperidin-3-ol.

Typically, for medical use, salts of the therapeutic agents will be pharmaceutically acceptable salts; although other salts may be used in the preparation of the compound or of the pharmaceutically acceptable salt thereof. By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art.

For instance, suitable pharmaceutically acceptable salts of compounds useful in the invention may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid.

For example, S. M. Berge et al. describe pharmaceutically acceptable salts in detail in J. Pharmaceutical Sciences, 1977, 66:1-19. The salts can be prepared in situ during the final isolation and purification of the therapeutic compound, or separately by reacting the free base function with a suitable organic acid. Representative acid addition salts include acetate, adipate, alginate, ascorbate, asparate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate; nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Also included within the scope of therapeutic agents are prodrugs. Typically, prodrugs will be functional derivatives of the therapeutic agents which are readily converted in vivo to the required (active) compound as active agents. Typical procedures for the selection and preparation of prodrugs are known to those of skill in the art and are described, for instance, in H. Bundgaard (Ed), Design of Prodrugs, Elsevier, 1985.

Single or multiple administrations of the therapeutic agents either alone or as a pharmaceutical composition can be carried out with dose levels and pattern being selected by the treating physician. Regardless, the therapeutic agents or pharmaceutical compositions should provide a quantity of the therapeutic agent sufficient to effectively treat the patient.

One skilled in the art would be able, by routine experimentation, to determine an effective, non-toxic amount of the therapeutic agent, or pharmaceutical composition containing the therapeutic agent, which would be required to treat or prevent the disorders and diseases. Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 11.0 mg to about 100 mg per kg body weight per 24 hours; about 1.0 mg to about 50 mg per kg body weight per 24 hours; about 11.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Further, it will be apparent to one of ordinary skill in the art that the optimal quantity and spacing of individual dosages of a therapeutic agent will be determined by the nature and extent of the condition being treated, the form, route and site of administration, and the nature of the particular vertebrate being treated. Also, such optimum conditions can be determined by conventional techniques.

It will also be apparent to one of ordinary skill in the art that the optimal course of treatment, such as, the number of doses of the therapeutic agent given per day for a defined number of days, can be ascertained by those skilled in the art using conventional course of treatment determination tests.

Whilst the therapeutic agent may be administered alone, it is generally preferable that it be administered as a pharmaceutical composition/formulation. In general pharmaceutical formulations may be prepared according to methods which are known to those of ordinary skill in the art and accordingly may include a pharmaceutically acceptable carrier, diluent and/or adjuvant. The carriers, diluents and adjuvants must be “acceptable” in terms of being compatible with the other ingredients of the formulation, and not deleterious to the recipient thereof.

Examples of pharmaceutically and veterinarily acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

In a preferred form, the pharmaceutical compositions comprise an effective amount of an active agent, together with a pharmaceutically acceptable carrier, diluent and/or adjuvant as shown in Example 2.

The pharmaceutical compositions may be administered by standard routes. In general, the compositions may be administered by the transdermal, intraperitoneal, intracranial, intracerebroventricular, intracerebral, oral, or parenteral (e.g., intravenous, intraspinal, subcutaneous or intramuscular) route. The compositions may be in the form of a capsule suitable for oral ingestion, or in an aerosol form suitable for administration by inhalation, such as by intranasal inhalation or oral inhalation.

For administration as an injectable solution or suspension, non-toxic parenterally acceptable diluents or carriers can include, Ringer's solution, isotonic saline, phosphate buffered saline, ethanol and 1,2-propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration of the capsule.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulfite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides, or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate, and the like.

The emulsions for oral administration may further comprise one or more emulsifying agents. Suitable emulsifying agents include dispersing agents as exemplified above, or natural gums such as guar gum, gum acacia or gum tragacanth.

The compositions for parenteral administration will commonly comprise a solution of an active agent or a cocktail thereof dissolved in an acceptable carrier, such as water, buffered water, 0.4% saline, and 0.3% glycine etc, wherein such solutions are sterile and relatively free of particulate matter.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The pharmaceutical compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The formulations in liposome form may contain, in addition to the therapeutic agent(s), stabilisers, preservatives, excipients and the like. The preferred lipids are the phospholipids and the phosphatidylcholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

The invention will now be described in greater detail by reference to specific Examples which should not be construed as in any way limiting the scope of the invention.

### EXAMPLES

### Example 1

Methods

Subjects

Fifty-two individuals with a clinical diagnosis of PD and fifty-one healthy controls without any indications of motor dysfunction and seven individuals with a clinical diagnosis of severe depression were recruited, with informed consent, in Sydney, Australia. The depression subjects represented a disease control group suffering a functional, but not degenerative change, in central dopaminergic function. The age range was 43 to 88 years and did not differ between the PD (means age 67.8±1.3), control (mean age 63.6±1.8), or depression subject (mean age 64.5±6.5 p=0.44). A clinical diagnosis of PD was made if the total score obtained from Part III Motor Examination of the Unified Parkinson Disease Rating Scale (Martinez-Martin, et al., 1994) was equal to five or above and the patient was levodopa responsive. No subject exhibited any evidence of related motor disorders or of dementia. A further group of twenty-seven patients with a clinical diagnosis of Parkinson's disease and thirty age- and sex-matched controls without any indications of motor dysfunction were recruited, with informed consent, in Bochum, Germany. The age range of the German subjects was 41 to 80 years and did not differ between the PD (mean age 65.5±1.0) and control subjects (mean age 66.2±1.8 p=0.88. Neuropathological confirmation for the clinical diagnosis of PD was not available for any patient in this study.

Collection and Preparation of Serum Samples

Blood samples were collected with consent from each subject and the sera isolated by centrifugation. Sera were frozen at −80° C. until use.

Isolation of Human Neuromelanin

NM was isolated according to the method of Zecca and Swartz (1993) from the SN of 10 human subjects from Germany (age range: 41-64 years) with no history of neurological or neurodegenerative diseases. The SN were dissected from the brain within 36 hours of death on a cool plate (−10° C.) and pooled in a glass-Teflon homogeniser. The samples were homogenised in 20 ml water and centrifuged at 12 000 g for 10 min. The resulting pellets were washed twice with 50 mM phosphate buffer (pH 7.4), then incubated in 50 mM Tris buffer (pH 7.4) containing 0.5 mg.ml⁻¹ SDS at 37° C. for 3 h, followed by a further 3 h incubation with the addition of 0.2 mg.ml⁻¹ proteinase K. The resulting pellets were pooled and consecutively washed with saline, water, methanol and hexane. The resulting dark pellet was incubated for three periods of 8 h each in 150 mM EDTA (pH 7.4) before being washed twice with water and finally dried under vacuum.

Preparation of Synthetic Neuromelanins

Synthetic dopamine melanin (DAM) was prepared according to a modification of the method of Ben-Shachar et al. (1991). Two mM DA (Sigma, USA) was incubated in 50 mM Tris buffer (pH 8) containing 0.1 mM CuSO₄ for one month at room temperature. The resulting liquid was centrifuged at 12 000 g for 15 min and the pellet was washed three times in double distilled water at 4° C. The resulting melanin was finally lyophilised to a dry powder. Dopamine melanin was also synthesised using 0.1 mM Fe₂(SO₄)₃ instead of copper (FeDAM). Other types of synthetic melanins were produced using 2 mM noradrenaline (NAM) or serotonin (5HT-Mel) instead of dopamine. All chemicals used for synthetic melanin production were purchased from Sigma, Australia.

Enzyme-Linked Immunosorbant Assay

The ELISA was a modification of that reported by Rowe et al. (1998). The antigen used was either human NM or synthetic melanin biotinylated using EZ-Link Sulfo-NHS-LC-Biotin (Pierce, USA) according to the manufacturer's instructions and was prepared by sonification in phosphate-buffered saline to produce a suspension of fine, homogenous granules at a concentration of 1 mg/ml. One hundred μL aliquots were dispensed into a 96-well Reacti-Bind NeutrAvidin with Superblock plate (Pierce, USA) sealed with an acetate sheet, protected from light and incubated at room temperature for 2 hours. The plate was washed three times with wash buffer consisting of 0.5% Tween 20 0.1% bovine serum Albumin in Tris-buffered saline (pH 7.2 22° C.). 100 μL human sera diluted 1:50 in the wash buffer was added to each sextet of wells and incubated for 0.5 hr at room temperature. Following the primary incubation, the plate was washed three times with wash buffer and 100 μL of horseradish peroxidase-conjugated goat antihuman IgG (Pierce, Rockford, USA) was added diluted to 1:10 000 in wash buffer and incubated for a further 0.5 hr. Following three further washes with wash buffer the bound signal was detected using citrated ABTS (0.3 mg/ml 2-2′-azino-bis(3-ethylbenzthiazoline-6-sulfonic acid) activated with 0.03% hydrogen peroxide in 0.1 M sodium citrate pH 4.5. Following an hour incubation at room temperature absorbance was determined at 405 nm using a microplate reader (BioRad Benchmark, USA). Absorbance for each sample was measured in six wells, corrected relative to wells containing no antigen and the mean absorbance calculated. Each plate contained positive control samples consisting of pre- and anti-immune sera samples raised against isolated human NM in the rabbit amplified by anti-rabbit IgG (Pierce, Rockford, USA) as the secondary antibody.

Synthetic dopamine melanin was replaced by isolated human NM prepared in the same manner in some experiments.

Analysis

Absorbance results and effect of sex on absorbance were analysed using the Mann Whitney U test. Cross-reactivity was analysed using one-way analysis of variance while the effect of age on absorbance was analysed using regression analysis.

Results

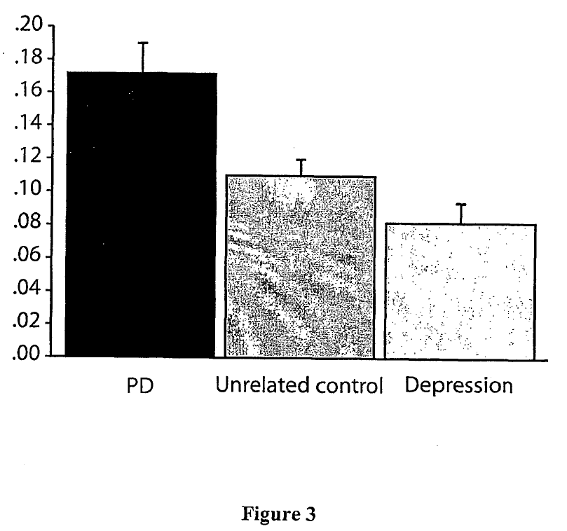

Results from the Sydney study are shown in FIG. 3 and demonstrate that sera obtained from persons with a clinical diagnosis of PD (mean 0.171±0.020) exhibits a significantly increased immune response on the ELISA test compared with sera obtained from age- and sex-matched healthy control individuals (mean 0.108±0.011, p=0.005) and from individuals suffering from depression (mean 0.080±0.013, p=0.046) using synthetic DAM as the antigen. A similar response was seen in the German study where the immune response of the PD patients (mean 0.109±0.007) was significantly higher (p=0.001) than the controls (mean 0.079±0.003).

The specificity of the immune response was determined by testing the cross-reactivity of the sera for various substrates. The significantly different absorbance response stimulated by exposure of parkinsonian compared with control sera to DAM was also exhibited when FeDAM or NAM were used as the antigen (both p<0.001). In contrast, no difference in absorbance was recorded when 5HT-Mel was used as the assay antigen (p=0.64, FIG. 3). Sera obtained from 48 healthy individuals aged from 21 to 92 years demonstrated the absorbance response was not significantly affected by the age (p=0.10) or sex (p=0.12) of the serum donor.

Discussion

The present invention provides a new and inexpensive method to identify an immune response in PD patients to DAM and isolated human NM. This response indicates the release of NM from central dopaminergic neurons and thus the death of neurons within the central dopaminergic system. Such a test could confirm a clinical diagnosis of PD or indicate the presence of preclinical PD in cases where clinical symptomology are not yet present. The results demonstrate that an enhanced IgG response is associated with a clinical diagnosis of PD and that the response is specific for catecholaminergic-based melanins. A slightly enhanced IgG response in PD patients was also identified in assays in which DAM was replaced by NAM as the antigen. This response is consistent with the loss of the noradrenergic neurons of the locus ceruleus in PD as these neurons also form a melanin based upon noradrenaline, rather than dopamine, which is thought to be structurally similar to NM (Double et al., 1999). It is unknown whether the increased response to NAM resulted from a specific response from noradrenaline neuromelanin-stimulated IgG or a combined response to DAM and NAM. The locus coruleus, contains, however, only 40,000 pigmented cells compared with the approximately 400,000 pigmented cells within the SN, so that the magnitude of a specific immune response to the loss of noradrenaline neuromelanin is likely to be significantly less than that induced by the simultaneous death of dopaminergic cells of the SN.

Currently, no other method can reliably identify death of central dopaminergic cells in vivo. The identification of the release of NM from these cells by the method of the present invention represents a specific and sensitive technique to determine neurodegeneration of central dopaminergic systems.

The present inventors discovered that the detection of the release of NM occurring at cell death would form a specific marker for the death of these melanised cells and thus for the disorders characterised by the death of these cells, even prior to the onset of symptoms. The method disclosed herein provides a means of detecting a humoral IgG response to the release of NM from dying cells which utilises the enzyme-linked immunosorbant assay (ELISA) and either NM isolated from the human brain or synthetic dopamine melanin as the antigen. This assay demonstrated that a humoral response to NM can be detected in a peripheral tissue (blood) and that this response is significantly enhanced in patients suffering from clinical symptoms of PD.

Another element which also may stimulate an immune response in PD is Lewy Bodies (LB). LB are pathological inclusions which form inside the dopaminergic-neurons in PD and, like NM, are released upon death of these cells. LB contain modified forms of some proteins (such as synuclein and neurofilaments) which aggregate into round inclusion bodies. Detection of antibodies to released LB may also be a means of is detecting a neurodegenerative disease in a subject in the clinical or pre-clinical phase of said neurodegenerative disease.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### Example 2

### Pharmaceutical Formulations

The therapeutic agent(s) for use in the present invention may be administered alone, although it is preferable that they be administered as a pharmaceutical formulation. The active ingredient may comprise, from 0.001% to 10% by weight, and more typically from 1% to 5% by weight of the formulation, although it may comprise as much as 10% by weight.

Specific preferred pharmaceutical compositions are outlined below. However, the following are to be construed as merely illustrative examples of formulations and not as a limitation of the scope of the present invention in any way.

### Example 2(a)

### Composition for Inhalation Administration

For an aerosol container with a capacity of 20-30 ml: a mixture of 10 mg of zonisamide with 0.5-0.8% by weight of a lubricating agent, such as polysorbate 85 or oleic acid, is dispersed in a propellant, such as freon, and put into an appropriate aerosol container for either intranasal or oral inhalation administration.

### Example 2(b)

### Composition for Parenteral Administration

A pharmaceutical composition for intramuscular injection could be prepared to contain 1 mL sterile buffered water, and 1 mg of zonisamide.

Similarly, a pharmaceutical composition for intravenous infusion may comprise 250 ml of sterile Ringer's solution, and 5 mg of zonisamide.

### Example 2(c)

### Capsule Composition

A pharmaceutical composition in the form of a capsule may be prepared by filling a standard two-piece hard gelatin capsule with 50 mg of zonisamide, in powdered form, 100 mg of lactose, 35 mg of talc and 10 mg of magnesium stearate.

### Example 2(d)

### Injectable Parenteral Composition

A pharmaceutical composition of this invention in a form suitable for administration by injection may be prepared by mixing 1% by weight of zonisamide in 10% by volume propylene glycol and water. The solution is sterilised by filtration.

### REFERENCES

Ben-Shachar, D., Riederer, P. and Youdim, M. B. H., “Iron-melanin interaction and lipid peroxidation: implications for Parkinson's disease”, J. Neurochem., 57 (1991) 1609-1614.
Double, K., Riederer, P. and Gerlach, M., “Significance of neuromelanin for neurodegeneration in Parkinson's disease”, Drug News Dev, 12 (1999) 333-340.
Fearnley, J. and Lees, A., “Ageing and Parkinson's disease: substantia nigra regional selectivity”, Brain, 114 (1991) 2283-2301.
Halliday, G., McRitchie, D., Cartwright, H., Pamphlett, R., Hely, M. and Morris, J., “Midbrain neuropathology in idiopathic Parkinson's disease and diffuse Lewy body disease”, J Clin. Neurosci., 3 (1996) 52-60.
Martinez-Martin, P., Gil-Nagel, A., Balseiro Gomez, J., Morlan, Gracia, L., Martinez-Sarries, J., Bermejo, F. and The Cooperative Multicentre Group. “Unified Parkinson's disease Rating Scale Characteristics and Structure”. Mov. Disorders 9 (1994) 76-83.
Morrish, P., Rakshi, J., Bailey, D., Sawle, G. and Brooks, D., “Measuring the rate of progression and estimating the preclinical period of Parkinson's disease with (18F)dopa PET”., J Neurol Neurosurg Psychiatry, 64 (1998) 314-319.
Rowe, D., Le, W., Smith, G. and Appel, S., “Antibodies from patients with Parkinson's disease react with protein modified by dopamine oxidation”, J. Neurosci. Res., 53 (1998) 551-558.
Zecca, L. and Swartz, H. M., “Total and paramagnetic metals in human substantia nigra and its neuromelanin”, J. Neural Trans. Park. Dis. Sect., 5 (1993) 203-213.

1. A method for detecting a neurodegenerative disease in a subject, said method comprising testing the subject for an indicator of release of neuromelanin from cells in the brain, wherein a positive test is indicative of death of brain cells containing neuromelanin and is characterised by an elevated level of the indicator of release of neuromelanin compared to control values.

2. The method of claim 1, wherein the subject is tested prior to having any clinical symptoms of said neurodegenerative disease.

3. The method of claim 1, wherein said cell death is associated with the neurodegenerative disease in the subject.

4. The method of claim 1, wherein the neurodegenerative disease has clinical symptoms selected from the group consisting of: tremor, rigidity, bradykinesia and slowness of movement.

5. The method of claim 1, wherein the indicator is an immune response in the form of circulating antibodies to neuromelanin, or analogues thereof, in said subject.

6. The method of claim 5, wherein the detection method employs the detection of antibodies capable of reacting to neuromelanin, or an antigenic fragment or analogue thereof, present in said subject.

7. The method of claim 6, wherein the analogue of neuromelanin is selected from the group consisting of synthetic dopamine melanin and synthetic noradrenaline melanin.

8. The method of, claim 1 wherein said method comprises:
(a) isolating sera from a blood sample from said subject;
(b) incubating said sera with an antigen selected from human NM or synthetic dopamine melanin under conditions suitable for antibody-antigen binding; and
(c) detecting bound antibody.

9. A method of treatment of a neurodegenerative disease in a subject, said method comprising:
(a) screening subjects to detect a neurodegenerative disease using the method of claim 1, and
(b) initiating treatment for subjects tested positive for the indicator of release of neuromelanin from cells in the brain.

10. The method of claim 9, wherein said treatment comprises administering a therapeutically effective amount of at least one of the following: antioxidants, iron chelators, nonamine oxidase inhibitors, apoptosis inhibitors, growth factors, dopamine receptor inhibitors, zonisamide, benzamide compounds, ethanesulfonyl-piperidine derivatives, endogenous enzymes which protect against oxidative damage or inhibitors of excitatory damage, or a combination thereof.

11. The method of claim 10, wherein said benzamide compound is selected from the group consisting of: N-tert-butyl-4-acetamidobenzamide, N-iso-propyl-4-acetamidobenzamide, N-tert-amyl-4-acetamidobenzamide, N-tert-butyl-3-acetamidobenzamide, N-methylcyclopropyl-4-acetamidobenzamide, N-n-butyl-4-acetamidobenzamide, N-n-pentyl-2-acetamidobenzamide, N-tert-butyl-2-acetamidobenzamide, N-iso-butyl-4-acetamidobenzamide, N-n-propyl-4-acetamidobenzamide, N-n-propyl-4-acetamidobenzamide, N-1,2-dimethylpropyl-4-acetamidobenzamide, N-n-pentyl-4-acetamidobenzamide, N-2-methylbutyl-4-acetamidobenzamide, N-tert-butyl-2,3-diacetamidobenzamide, N-tert-amyl-2,4-diacetamidobenzamide, N-tert-butyl-2,5-diacetamidobenzamide, N-tert-butyl-2,6-diacetamidobenzamide, N-tert-butyl-3,4-diacetamidobenzamide, N-tert-butyl-3,5-diacetamidobenzamide, N-iso-propyl-4-nitrobenzamide, N-tert-butyl-3-nitrobenzamide, N-tert-butyl-2-nitrobenzamide, N-n-butyl-4-nitrobenzamide, N-n-propyl-4-nitrobenzamide, N-tert-butyl-3,5-nitrobenzamide, N-tert-amyl-4-nitrobenzamide, N-1,2-dimethylpropyl-4-nitrobenzamide, N-n-butyl-3-nitrobenzamide, N-n-butyl-3,5-dinitrobenzamide, N-methylcylopropyl-4-nitrobenzamide, N-n-butyl-2-nitrobenzamide, N-n-pentyl-4-nitrobenzamide, N-2-methylbutyl-4-nitrobenzamide, N-n-pentyl-2-nitrobenzamide, N-1-methylpropyl-4-nitrobenzamide, N-tert-butyl-3-aminobenzamide, N-tert-butyl-4-aminobenzamide and N-methylcylopropyl-4-aminobenzamide, or a combination thereof.

12. The method of claim 10, wherein said ethanesulfonyl-piperidine derivative is selected from the group consisting of 4-[-2-(4-benzyl-piperidine-1-yl)-ethanesulfonyl]-phenol; 4-[2-(4-p-tolyloxy-piperidine-1-yl)-ethanesulfonyl]-phenol; (−)-(3R,4R)- or (3S,4S)-4-benzyl-1-[2-(4-hydroxy-benzenesulfonyl)-ethyl]-piperidin-3-ol; (+)-(3S,4S)- or (3R,4R)-4-benzyl-1-[2-(4-hydroxy-benzenesulfonyl)-ethyl]-piperidin-3-ol; (3RS,4RS)-4-benzyl-1-[2-(4-hydroxy-benzenesulfonyl)-ethyl]-piperidin-3-ol; (−)-(3R,4R)- or (3S,4S)-1-[2-(4-hydroxy-benzenesulfonyl)-ethyl]-4-(4-methyl-benzyl)-piperidin-3-ol; (+)-(3R,4R)- or (3S,4S)-1-[2-(4-hydroxy-benzenesulfonyl)-ethyl]-4-(4-methyl-benzyl)-piperidin-3-ol; and (3RS,4RS)-1-[2-(4-hydroxy-benzenesulfonyl)-ethyl]-4-(4-methyl-benzyl)-piperidin-3-ol, or a combination thereof.

13. The method of claim 10, wherein said endogenous enzymes which protect against oxidative damage are selected from the group consisting of: glutathione, superoxide dismutase and catalase, or a combination thereof.

14. The method of claim 10, wherein said treatment comprises administering a therapeutically effective amount of zonisamide.

15. The method of claim 1, wherein the neurodegenerative disease is selected from the group consisting of idiopathic Parkinson's disease, and parkinsonism including Multisystem Atrophy, Progressive Supranuclear palsy, Pick's disease, Corticobasal Degeneration and Dementia with Lewy Bodies.

16. The method of claim 15, wherein the neurodegenerative disease is idiopathic Parkinson's disease.

17. A system for the detection of a neurodegenerative disease in a subject, said system comprising:
(a) means for capturing an indicator of release of neuromelanin from cells in the brain of a subject; and
(b) means for detecting the captured indicator of release of neuromelanin from cells in the brain.

18. The system of claim 17, wherein said indicator of release of neuromelanin is circulating antibodies to neuromelanin, or analogues thereof, in said subject.

19. The system of claim 17, wherein said means for capturing the indicator of release of neuromelanin is neuromelanin or an antigenic fragment or analogue thereof immobilized to a solid surface.

20. The system of claim 17, wherein said system comprises neuromelanin or an antigenic fragment or an analogue thereof, bound to a solid support and a source of a detectably labelled probe.

21. The system of claim 20, wherein said probe is a labelled antihuman IgG.

22. The system of claim 20, wherein said probe is horseradish peroxidase-conjugated goat antihuman IgG.

23. The system of claim 17, wherein said system is an ELISA-based system.

24. The system of claim 17, wherein said system is provided in kit form.

25. The system of claim 17, wherein the neurodegenerative disease is selected from the group consisting of idiopathic Parkinson's disease, and parkinsonism including Multisystem Atrophy, Progressive Supranuclear palsy, Pick's disease, Corticobasal Degeneration and Dementia with Lewy Bodies.

26. The system claim 24, wherein the neurodegenerative disease is idiopathic Parkinson's disease.

27. A method for detecting a neurodegenerative disease in a subject comprising testing the subject for an indicator of release of neuromelanin from cells in the brain, said method comprising
(a) contacting a serum sample of said subject to a detection system comprising neuromelanin, or an antigenic fragment or an analoque thereof, bound to a solid support,
(b) adding sample of the detectably labelled probe that binds the indicator of release of neuromelanin from cells in the brain, and
(c) detecting probe bound antibody within said serum sample, wherein a positive test is indicative of death of brain cells containing neuromelanin and is characterised by an elevated level of the indicator of release of neuromelanin compared to control values.

28. The method of claim 27, wherein the neurodegenerative disease is selected from the group consisting of idiopathic Parkinson's disease, and parkinsonism including Multisystem Atrophy, Progressive Supranuclear palsy, Pick's disease, Corticobasal Degeneration and Dementia with Lewy Bodies.

29. The method of claim 27, wherein the neurodegenerative disease is idiopathic Parkinson's disease.

30. The method of claim 27, wherein said subject is tested for the presence of said neurodegenerative disease prior to having any clinical symptoms of said neurodegenerative disease.
